(19) United States
(12) Patent Application Publication
(10) Pub. No.: US 2024/0269640 A1
(43) Pub. Date: Aug. 15, 2024

(54) METHODS AND COMPOSITIONS FOR LARGE-SCALE CONJUGATABLE POLYMER AND PROTEIN SYNTHESIS

(71) Applicant: Ligandal, Inc., Brooklyn, NY (US)

(72) Inventor: Andre Watson, Brooklyn, NY (US)

(21) Appl. No.: 18/496,770

(22) Filed: Oct. 27, 2023

Related U.S. Application Data
(63) Continuation of application No. PCT/US2022/072027, filed on Apr. 29, 2022.
(60) Provisional application No. 63/182,176, filed on Apr. 30, 2021.

(51) Int. Cl. B01J 19/00; C07K 1/04

(52) U.S. Cl. CPC B01J 19/0046; C07K 1/045; B01J 2219/00006; B01J 2219/00051; B01J 2219/00186; B01J 2219/00306; B01J 2219/00353; B01J 2219/0072

(57) ABSTRACT

Methods and compositions for manufacturing large-scale quantities of conjugatable peptides/peptoids/polymers/nucleic acids and conjugatable proteins, as well as hybrid materials consisting of synthetic and unnatural amino acids, glycopeptides, proteoglycans, and other molecular modifications are disclosed, for a variety of purposes including rapid antidote and vaccine applications in biodefense, therapeutics, diagnostics, theranostics, thin films, multilayered assemblies, biofilms, sensors, drug delivery vehicles, gene delivery vehicles, gene editing vehicles, staged release compounds, and the like.

### CROSS-REFERENCE TO RELATED APPLICATIONS

This application is a continuation of PCT International Application No. PCT/US2022/072027, filed on Apr. 29, 2022, which claims the benefit of U.S. Provisional Patent Application No. 63/182,176, filed Apr. 30, 2021, which is incorporated herein by reference in its entirety.

### BACKGROUND

Currently available systems are limited by slow, costly synthesis processes; inefficient scale-up and purification; difficulty of manufacturing longer amino acid sequences via synthetic means; and difficulty of tethering recombinantly-synthesized proteins to synthetic scaffolds. These issues are compounded by the incompatibility of synthetic technologies with longer sequence synthesis needs, and incompatibility of recombinant technologies with facile techniques for tethering beyond His-tags, snoop-tags, snap-tags, C-tags, non-specific reactive chemistries, and other mechanisms that are limited in application to broad substrate materials. Similarly, while synthetic peptide chemistries allow for tailoring of structures and sequence and construction of unnatural polymer compositions, and the like; these synthetic peptides are limited in size due to constraints of synthesis, difficulty of purification of longer sequences, and difficulty in recreating folding structures of larger proteins.

Current peptide synthesis approaches, including close-looped fluidic-based approaches, generate extensive waste product including dimethylformamide (DMF), N-methylpyrrolidone, HCTU, HBTU, and other solvents/coupling reagents.

Typical solvents used in solid phase peptide synthesis (SPPS) are wasted following synthesis, leading to large volumes of DMF and N-methylpiperidine disposal.

To overcome these limitations in the prior technology, the present disclosure proposes several critical innovations necessary for 1) overcoming cost issues of scaling large amounts of materials to production and 2) synthesizing and properly folding large protein structures while maintaining flexible conjugation chemistries to a variety of substrates.

### SUMMARY

The present disclosure relates to methods and compositions for manufacturing large-scale quantities of conjugatable peptides/peptoids/polymers/nucleic acids and conjugatable proteins, as well as hybrid materials consisting of synthetic and unnatural amino acids, glycopeptides, proteoglycans, and other molecular modifications, for a variety of purposes including rapid antidote and vaccine applications in biodefense, therapeutics, diagnostics, theranostics, thin films, multilayered assemblies, biofilms, sensors, drug delivery vehicles, gene delivery vehicles, gene editing vehicles, staged release compounds, and the like.

### BRIEF DESCRIPTION OF THE DRAWINGS

FIG. 1 depicts a design schematic showing one exemplary configuration of a peptide robot according to the present disclosure. At left, a photo is shown with Applicant's peptide synthesis robot. In the middle is shown a mockup of solution collection vessels, and at right is an annotated distillation column.

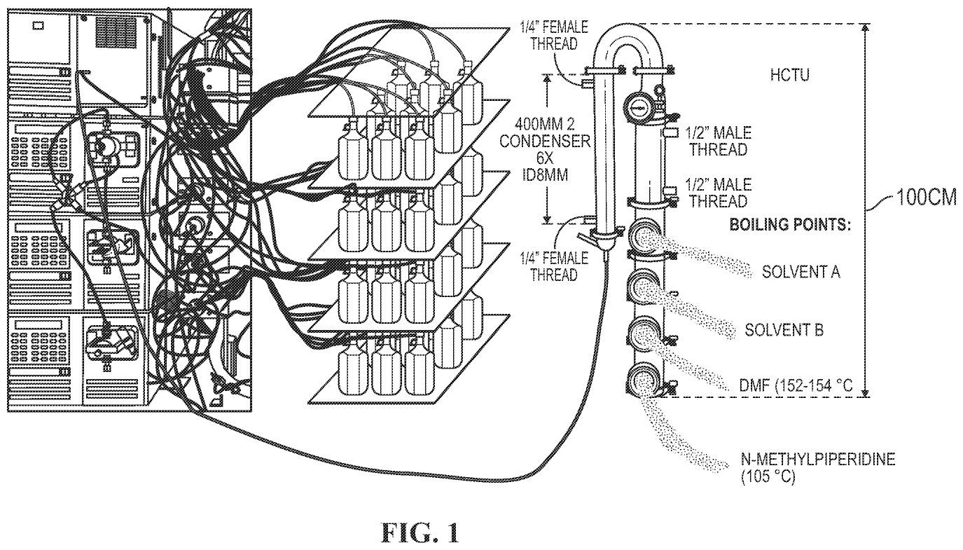

FIG. 2 depicts a schematic overview of apparatus and method overviews according to the present disclosure.

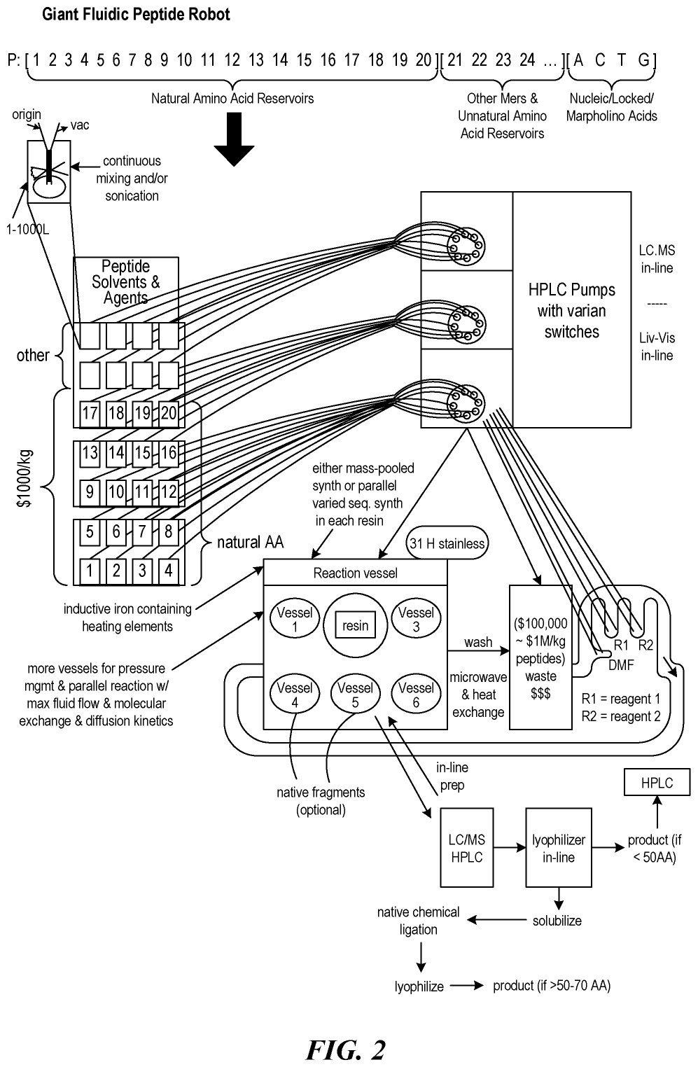

FIG. 3 depicts schematic representations of staple peptides and folding domains according to the present disclosure.

FIG. 4 depicts a schematic overview and outline of exemplary staple peptides according to the present disclosure.

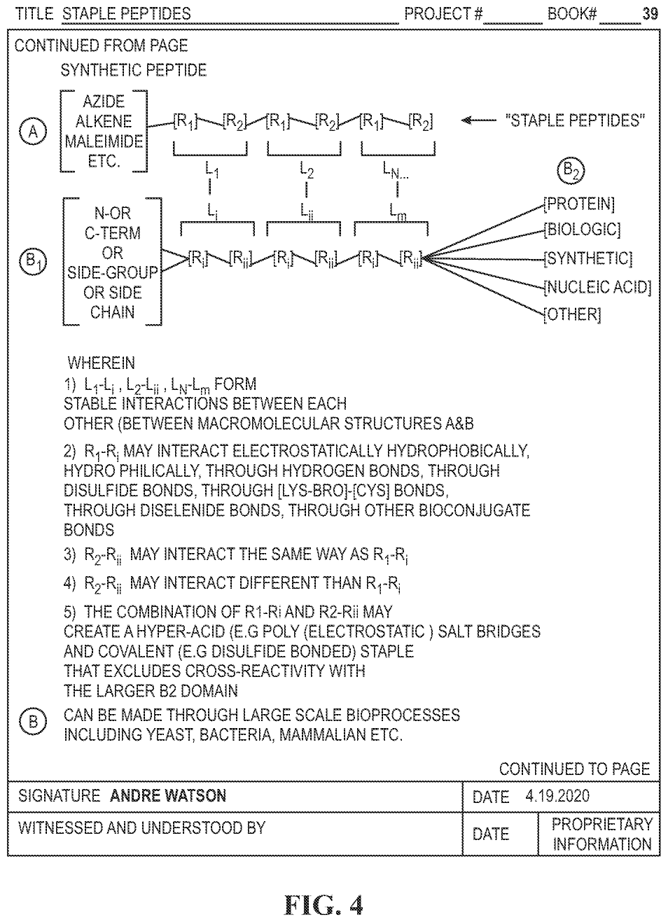

### DETAILED DESCRIPTION

The present disclosure relates to methods and compositions for large-scale conjugatable polymer and protein synthesis. In certain embodiments, the disclosure relates to an apparatus for large-scale conjugatable polymer and protein synthesis.

### Recycling Apparatus and Methods

In one aspect, the present disclosure relates to an apparatus for generating a conjugatable polymer, comprising (i) a plurality of reservoirs for holding a reaction fluid, (ii) a conduit for transporting the reaction fluid to a reaction chamber, the reaction chamber having a solid support, wherein a polymer product is synthesized on the solid support, (iii) a conduit for transporting the reaction fluid from the reaction chamber to a used reagent collection chamber, (iv) a conduit for transporting the reaction fluid from the used reagent collection chamber to a distillation component, the distillation component having a heating element, and (v) a recycled reagent collection chamber. In certain embodiments, the present disclosure relates to an apparatus having a plurality of reservoirs for holding a reaction fluid. In certain embodiments, the apparatus includes a first reservoir, wherein the first reservoir holds a first reaction fluid comprising an amino acid. The apparatus additionally comprises a first conduit for transporting the first reaction fluid from the first reservoir to a first reaction vessel having a support for attaching an amino acid chain. The amino acid chain is formed by sequentially adding a reaction fluid comprising desired amino acid to the reaction chamber.

In certain aspects, an apparatus according to the present disclosure comprises a plurality of reservoirs for holding a reaction fluid. In certain embodiments, the reaction fluid comprises a nucleic acid, a locked nucleic acid (LNA), or a morpholino.

In certain embodiments, the reaction mixture includes a coupling reagent. In some embodiments, the coupling reagent is an aluminum coupling reagent, e.g., O-(1H-6-Chlorobenzotriazole-1-yl)-1,1,3,3-tetramethyluronium hexafluorophosphate (HCTU), 2-(1H-benzotriazol-1-yl)-1,1,3,3-tetramethyluronium hexafluorophosphate (HBTU), O-(Benzotriazol-1-yl)-N,N,N′,N′-tetramethyluronium tetrafluoroborate (TBTU), or O-(7-Azabenzotriazol-1-yl)-N,N,N′,N′-tetramethyluronium tetrafluoroborate (TATU). Coupling reagents suitable for use with the present disclosure include, but are not limited to (Benzotriazol-1-yloxy)tris(dimethylamino)phosphonium hexafluorophosphate (BOP), (Benzotriazol-1-yloxy)tripyrrolidinophosphonium hexafluorophosphate (PyBOP), (7-Azabenzotriazol-1-yloxy)tripyrrolidinophosphonium hexafluorophosphate (PyAOP), Bromotripyrrolidinophosphonium hexafluorophosphate (PyBrOP), BOP-Cl, O-[(Ethoxycarbonyl)cyanomethylenamino]-N,N,N′,N′-tetra methyluronium tetrafluoroborate (TOTU), C₁₂H₁₉F₆N₄O₄P (COMU), O—(N-Suc-cinimidyl)-1,1,3,3-tetramethyl-uronium tetrafluoroborate (TSTU), O-(5-Norbornene-2,3-dicarboximido)-N,N,N′,N′-tetramethyluronium tetrafluoroborate (TNTU), O-(1,2-Dihydro-2-oxo-1-pyridyl-N,N,N′,N′-tetramethyluronium tetrafluoroborate (TPTU), N,N,N′,N′-Tetramethyl-O-(3,4-dihydro-4-oxo-1,2,3-benzotriazin-3-yl)uranium tetrafluoroborate (TDBTU), N,N,N′N′-Tetramethyl-O—(N-succinimidyl)uronium tetrafluoroborate (TSTU), 2-(5-Norborene-2,3-dicarboximido)-1,1,3,3-tetramethyluronium tetrafluoroborate (TNTU), 2-(2-Pyridon-1-yl)-1,1,3,3-tetramethyluronium tetrafluoroborate (TPTU), 3-(Diethylphosphoryloxy)-1,2,3-benzotriazin-4(3H)-one (DEPBT), Carbonyldiimidazole (CDI), N,N,N′,N′-Tetramethylchloroformamidinium Hexafluorophosphate (TCFH), and the like.

In certain aspects, an apparatus according to the present disclosure comprises a conduit for transporting the reaction fluid to a reaction chamber, the reaction chamber having a solid support. In certain embodiments, the support comprises a resin. This support can also be a different substrate, e.g., including gold, gold nanoparticles, other plasmonic surfaces, and other chip-based sensor technologies that may be introduced to various biosensors without the need for separation from the support substrate.

In certain aspects, an apparatus according to the present disclosure comprises a conduit for transporting the reaction fluid from the reaction chamber to a used reagent collection chamber, and a conduit for transporting the reaction fluid from the used reagent collection chamber to a distillation component, the distillation component having a heating element. In certain embodiments, the heating element heats the distillation component to a specified temperature to separate the reagents in the reaction mixture for future use. For example, this distillation process is utilized to separate dimethylformamide (153° C. boiling point), N-methylpiperidine (105° C. boiling point), dichloromethane (39.6° C. boiling point), chloroform (61.2° C. boiling point), acetonitrile (82° C. boiling point), hexafluoro-2-propanol (58.2° C. boiling point), ether (35° C. boiling point), acetone (56° C. boiling point), methanol (65° C. boiling point), tetahydrofuran (66° C. boiling point), hexane (69° C. boiling point), ethyl acetate (77° C. boiling point), N,N-diisopropylethylamine (127° C. boiling point), hydrazine (114° C. boiling point), TFA (72.4° C. boiling point), pyrazole-1-carboxamide (186-188° C. boiling point), or water (100° C. boiling point), toluene (111° C. boiling point), pyridine (115° C. boiling point), acetic acid (118° C. boiling point), dimethylsulfoxide (189° C. boiling point), or another reaction solvent reasonably understood to be usable by one ordinarily skilled in the art for peptide, peptoid, glycan, proteoglycan, glycoprotein, nucleic acid, or LNA/MNA/PNA synthesis from one or more reaction fluids in series or parallel for subsequent re-use. These fluids may be stored in a recycled reagent collection chamber or they may be cycled through the apparatus for a subsequent reaction without storage.

In certain embodiments, the apparatus additionally comprises an in-line purification component. The in-line purification component may be, for example, a high-performance liquid chromatography (HPLC) system. In certain embodiments, the HPLC system includes a plurality of pumps and a plurality of varian switches. In certain embodiments, the present disclosure relates to methods for fragmental peptide synthesis (FPS), wherein an amino acid chain is synthesized in short (e.g., less than 30 amino acids) segments and purified for subsequent assembly into a larger amino acid product. The amino acid chains can then be assembled into the larger amino acid product using traditional bioconjugation techniques known in the art, e.g., native chemical ligation (NCL). Additional embodiments include an inline liquid chromatography coupled mass spectroscopy (LCMS) component or ultraviolet visible (UV-vis) spectroscopy component.

In certain embodiments, the apparatus additionally comprises an in-line lyophilization component. For example, the in-line lyophilization component may be used to lyophilize a polymer product at varying stages of synthesis.

### Staple Peptides

In certain aspects, the present disclosure relates to compositions and methods for generating a polymer product coupled to a target using a synthetic “staple.” In certain embodiments, the polymer products are recombinant proteins generated using recombinant protein synthesis with synthetic staples for facile coupling to a variety of surfaces and substrates (hereinafter “SYNTHPRO”).

Current peptide synthesis approaches introduce compounding errors as the number of amino acids in a sequence increase, and the difficulty of purifying an ideal mass fraction increases as the peptide lengthens.

By synthesizing fragments <30 AA, or as small as 10 AA, it is possible to purify to 98-99% purity and then perform assembly. In-line mass spectrometry, HPLC, FPLC and the like can enable high-purity fragments to be rapidly generated, and then assembled into larger sequences. As an illustrative example, >99% purity sequences may yield ˜93% purity when assembled together, with the resulting sequence being easier to purify to 99% purity than if the entire sequence had been made, resulting in <50% purity.

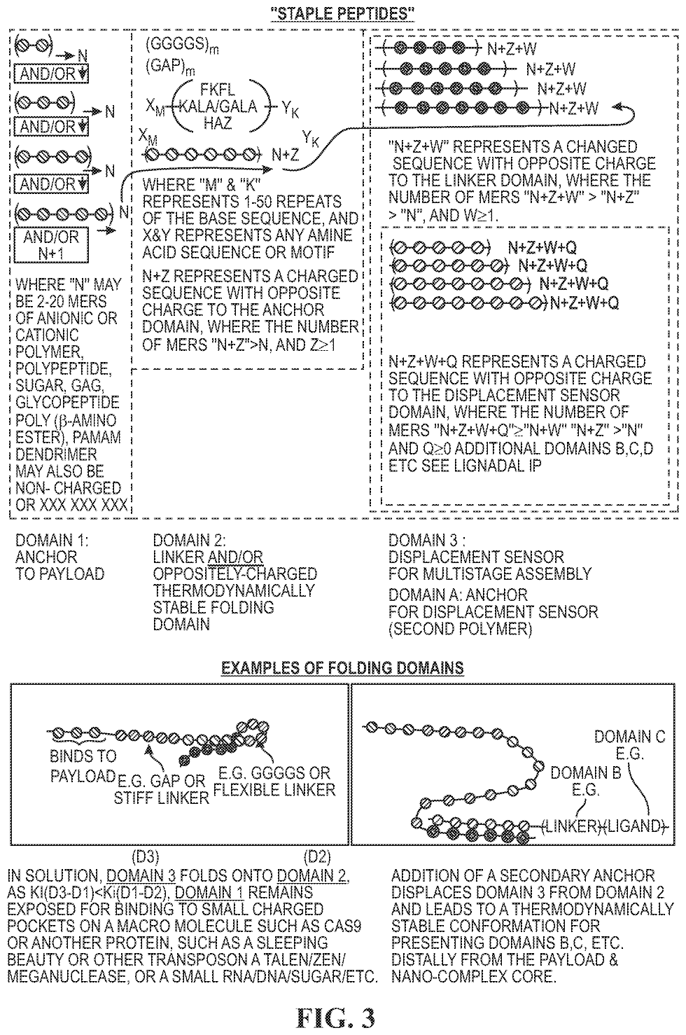

In certain embodiments provided herein, recombinant protein synthesis is achieved using a synthetic staple to facilitate coupling of a protein to a target substrate. In certain embodiments, the target substrate is a protein, a synthetic product, a nucleic acid, or a biologic product. See, e.g., FIG. 3.

A full range of synthetic peptides, peptoids, nucleic acids, LNAs, MNAs, PNAs, PEGs, poly(β-amino esters), sugars, dimers, trimers, oligomers, and other synthetic polymers may be synthesized, assembled, or modified with “staple” techniques that complement a specific sequence on a recombinant protein, and then the synthetic-recombinant or synthetic SYNTHPRO may be further conjugated to a variety of surfaces via azide-alkene cycloaddition, maleimide, isothiocyanate, isocyanate, acyl azide, NHS ester, sulfonyl chloride, tosylate ester, aldehyde and glyoxal, epoxide and oxirane, carbonate, arylation, imidoester, carbodiimide, anhydride, fluorophenyl ester, hydroxymethyl phosphine derivative, amide guanidination, haloacetyl, alkyl halide, aziridine, acryloyl, vinyl sulfone, metal-thiol, diazoalkane, diazoacetyl, N,N′-carbonyl diimidazole, hydrazine, hydrazide, Schiff base formation, reductive amination, aminooxy derivative, Mannich condensation, diazonium, iodination, aryl azide, benzophenone, anthraquinone, diazo, diazirine, psoralen, Diels-Alder, boronic acid complex formation, EDC coupling, EDC and sulfo-NHC, CMC, DCC<DIC, Woordward's Reagent K, homobifunctional NHS ester, homobifunctional imidoester, homobifunctional sulfylhydryl reactive crosslinker, difluorobenzene, homobifunctional photoreactive crosslinker, homobifunctional aldehyde, BIS-Epoxide, homobifunctional hydrazide, BIS-diazonium, BIS-alkylhalide, amine-reactive and sulfylhydryl-reactive crosslinkers, carbonyl reactive and sulfylhydryl reactive crosslinkers, amine-reactive and photoreactive crosslinkers, sulfylhydryl-reactive and photoreactive crosslinkers, carbonyl-reactive and photoreactive crosslinkers, carboxylate-reactive and photoreactive crosslinkers, arginine-reactive and photoreactive crosslinkers, trifunctional crosslinkers, and the like.

These chemistries, many of which are either not possible with recombinant technologies or are non-specific for a given portion of the recombinant protein's sequence, are enabled by the combination of synthetic staples with recombinant proteins.

A staple allows for a combination of avidity-generating and covalent-bond-forming sequences to interact, such as through interaction of aliphatic chains, hydrophilic chains, electrostatically opposite chains, and other chains exhibiting hydrogen bonding or van der Waals forces that are preferentially avid for two specific interacting motifs versus the bulk protein complexes.

In one such embodiment, a sequence such as ECECECECEC or EEECCCEEEE may interact with RCRCRCRC or RRRCCCRRRR, and similar sequences, whereby the negative and positive charges form a preferentially avid interaction that subsequently leads to disulfide bond formation. In certain embodiments, one or more of the chains are synthetic. In these embodiments, alternative chemistries such as Lys-Bro-Cysteine and other covalent bond approaches may be used. Covalent bonds as well as hydrogen bonds, hydrophobic interactions, hydrophilic interactions, and electrostatic interactions may be utilized to facilitate interaction of two chains with affinity for each other as well as subsequent reaction between the chains, and these interactions need not be electrostatic in their affinity for each other, whereby the stapling of two chains may happen covalently or through an enzymatic intermediary step between two sequences that are ligated together.

### Example 1: Peptide Synthesis and Reagent Recycling Methods

As proof of concept, applicant will utilize the apparatus and methods according to the present disclosure to synthesis peptides that can be used to test the binding affinity of a novel coronavirus spike protein for the angiotensin-converting enzyme 2 (ACE2). Sample components are summarized in Table 1.

TABLE 1

| | |
|---|---|
| Sample Type | 1) 6 peptides |
| (ie. protein, peptide, lipid, nucleic | 2) 3 proteins |
| acid, etc.) | 3) Convalescent antibodies |
| | (not immobilized) |
| Sample full name and | 1) LGDL-001 (PCT/US17/66545) |
| abbreviations | 2) LGDL-002 (PCT/US14/57000) |
| | 3) LGDL-003 (PCT/US17/66541) |
| | 4) LGDL-004 (PCT/US17/66545) |
| | 5) LGDL-005 (PCT/US19/29000) |
| | 6) LGDL-006 (PCT/US19/28004) |
| | 7) His-hACE2 |
| | 8) His-hCD147 |
| | 9) His-SPIKE-S1 |
| | 10) Antibody-RBD-Neutralizing-IgG-1 |
| | (RBD-Ab-1) |
| | 11) Antibody-RBD-Neutralizing-IgG-2 |
| | (RBD-Ab-2) |
| | 12) Convalescent antibody cocktail? |
| Molecular Weight | 1) Peptides: range from 7000 to 9700 Da |
| (Da, kDa or diameter in nm) | 2) His-hACE2: ~92,463 Da |
| | 3) His-hCD147: ~21,600 Da |
| | 4) His-Spike-S1: ~764,500 Da |
| | 5) RBD-Ab-1: ~150,000 Da |
| | 6) RBD-Ab-2: ~150,000 Da |
| | 7) CONV-AB IgA Abs: ~162,000 Da |
| | 8) CONV-AB IgM Abs: ~950,000 Da |
| | 9) CONV-AB IgG Abs: ~146,000 Da |
| | 10) CONV-AB IgE Abs: ~190,000 Da |
| Synthetic or recombinant? | 1) Peptides are synthetic |
| (If applicable for this binding partner, | 2) All other proteins are recombinant |
| specify) | 3) Convalescent antibodies are derived |
| | from recovered patient serum |
| Purity | Peptides: >90% via LC-MS |
| (specify how purity was determined, | His-hCD147: >97% via SDS-PAGE |
| e.g. SDS PAGE or SEC, MS, etc.) | His-hACE2: >95% via SDS-PAGE |
| Note: please ensure components are | His-Spike-S1: >90% via SDS-PAGE, >95% |
| as pure as possible e.g. >95%, | via SEC-HPLC |
| ideally >99%. | RBD-Ab-1: >95% via SDS-PAGE |
| | RBD-Ab-2: >95% via SDS-PAGE |
| | Convalescent antibodies: Diverse; we don't |
| | need to measure their specific presence, so |
| | much as their bulk displacement when |
| | peptides are flown over the immobilized |
| | spikes, as well as their stickiness to the |
| | immobilized peptides |
| Method of purification? | See above. |
| (Please provide as much detail as | |
| possible) | |
| Isoelectric Point | |
| (If applicable for this binding partner, | |
| specify) | |
| Presence of tags? | Peptides: alkyne, but can conjugate to |
| (His, Biotin etc.; please specify) | another linker if necessary |
| OR Presence of free amine, | Recombinant proteins: HIS tag |
| carboxyl or thiol groups? | Antibodies: Fc domain |
| (Please specify) | |
| Stock Concentration | All compounds not noted below are |
| (mM or mg/mL) | lyophilized. |
| | 1) One antibody is at a 1 mg/ml |
| | concentration |
| | 2) Convalescent serum would be |
| | unknown concentration |
| Matrix Composition | Can use HEPES or PBS, whatever works |
| (of the stock solution) | best. |
| | The peptides contain TFA. |
| | The recombinant proteins contain 5-8% |
| | trehalose and mannitol, and 0.01% |
| | Tween80 as protectants |

The following patent applications listed in Table 1 are incorporated herein by reference: PCT/US14/57000, PCT/US17/66541, PCT/US17/66545, PCT/US19/29000, and PCT/US19/28004.

Binding affinity testing for the present Example will be conducted as summarized below:
1. Immobilize receptor of interest on chip, flow relevant native biological or known binding partner over chip (i.e., support) to create binding, then flow peptides, peptoids, or synthesized binding agents over chip to measure displacement of spike protein.
2. Immobilize relevant native biological or known binding partner on chip, then flow corresponding receptor or secondary binding analyte over chip to saturation concentration. Then introduce peptides, peptoids, or synthesized binding agents to measure displacement of binding partner from corresponding receptor or secondary binding analyte.
3. Immobilize relevant native biological or known binding partner on chip, then flow each antibody over chip to saturation concentration. Then introduce peptides to measure displacement of antibodies from relevant native biological or known binding partner.
4. Immobilize relevant native biological or known binding partner on chip, then flow corresponding receptor or secondary binding analyte over chip to saturation concentration. Then flow antibodies over chip to determine dissociation EC50 of relevant native biological or known binding partner with respect to corresponding receptor or secondary binding analyte.
5. Immobilize relevant native biological or known binding partner on chip, then flow corresponding receptor or secondary binding analyte over chip to saturation concentration. Then flow peptides, peptoids, or synthesized binding agents over chip to displace relevant native biological or known binding partner, followed by flowing antibodies over chip to demonstrate enhanced binding of antibodies to relevant native biological or known binding partner following peptides, peptoids, or synthesized binding agents scavenging of corresponding receptor or secondary binding agent.
6. Immobilize each peptide, peptoid, or synthesized binding agent on a chip, then flow corresponding receptor or secondary binding analyte over chip to saturation concentration. Then introduce relevant native biological or known binding partner and measure displacement of peptides, peptoids, or synthesized binding agents from chip.
   [Above, “antibodies” may also be a tertiary or alternative binding agent with competitive binding for the given native biological or known binding partner, or its corresponding receptor or secondary binding analyte.]

Following synthesis of a given compound for testing, Applicant will utilize distillation to purify and recycle reagents from the experiment.

1. An apparatus for generating a conjugatable polymer, comprising (i) a plurality of reservoirs for holding a reaction fluid, (ii) a conduit for transporting the reaction fluid to a reaction chamber, the reaction chamber having a solid support, wherein a polymer product is synthesized on the solid support, (iii) a conduit for transporting the reaction fluid from the reaction chamber to a used reagent collection chamber, (iv) a conduit for transporting the reaction fluid from the used reagent collection chamber to a distillation component, the distillation component having a heating element, and (v) a recycled reagent collection chamber.

2. The apparatus of claim 1, wherein the apparatus comprises a plurality of reservoirs for holding a reaction fluid.

3. The apparatus of claim 2, wherein the apparatus comprises a first reservoir, wherein the first reservoir holds a first reaction fluid comprising an amino acid.

4. The apparatus of claim 3, further comprising a first conduit for transporting the first reaction fluid from the first reservoir to a first reaction vessel having a support for attaching an amino acid chain.

5. The apparatus of claim 4, wherein the amino acid chain is formed by sequentially adding a reaction fluid comprising desired amino acid to the reaction chamber.

6. (canceled)

7. The apparatus of claim 4, wherein the reaction fluid comprises a nucleic acid, a locked nucleic acid (LNA), or a morpholino.

8. The apparatus of claim 7, wherein the reaction mixture includes a coupling reagent.

9. The apparatus of claim 8, wherein the coupling reagent is an aluminum coupling reagent, e.g., O-(1H-6-Chlorobenzotriazole-1-yl)-1,1,3,3-tetramethyluronium hexafluorophosphate (HCTU), 2-(1H-benzotriazol-1-yl)-1,1,3,3-tetramethyluronium hexafluorophosphate (HBTU), O-(Benzotriazol-1-yl)-N,N,N′,N′-tetramethyluronium tetrafluoroborate (TBTU), or O-(7-Azabenzotriazol-1-yl)-N,N,N′,N′-tetramethyluronium tetrafluoroborate (TATU).

10. The apparatus of claim 8, wherein the coupling reagent is (Benzotriazol-1-yloxy)tris(dimethylamino)phosphonium hexafluorophosphate (BOP), (Benzotriazol-1-yloxy)tripyrrolidinophosphonium hexafluorophosphate (PyBOP), (7-Azabenzotriazol-1-yloxy)tripyrrolidinophosphonium hexafluorophosphate (PyAOP), Bromotripyrrolidinophosphonium hexafluorophosphate (PyBrOP), BOP-Cl, O-[(Ethoxycarbonyl)cyanomethylenamino]-N,N,N′,N′-tetra methyluronium tetrafluoroborate (TOTU), C₁₂H₁₉F₆N₄O₄P (COMU), O—(N-Suc-cinimidyl)-1,1,3,3-tetramethyl-uronium tetrafluoroborate (TSTU), O-(5-Norbornene-2,3-dicarboximido)-N,N,N′,N′-tetramethyluronium tetrafluoroborate (TNTU), O-(1,2-Dihydro-2-oxo-1-pyridyl-N,N,N′,N′-tetramethyluronium tetrafluoroborate (TPTU), N,N,N′,N′-Tetramethyl-O-(3,4-dihydro-4-oxo-1,2,3-benzotriazin-3-yl)uranium tetrafluoroborate (TDBTU), N,N,N′N′-Tetramethyl-O—(N-succinimidyl)uronium tetrafluoroborate (TSTU), 2-(5-Norborene-2,3-dicarboximido)-1,1,3,3-tetramethyluronium tetrafluoroborate (TNTU), 2-(2-Pyridon-1-yl)-1,1,3,3-tetramethyluronium tetrafluoroborate (TPTU), 3-(Diethylphosphoryloxy)-1,2,3-benzotriazin-4(3H)-one (DEPBT), Carbonyldiimidazole (CDI), or N,N,N′,N′-Tetramethylchloroformamidinium Hexafluorophosphate (TCFH).

11. The apparatus of claim 1, wherein the apparatus comprises a conduit for transporting the reaction fluid to a reaction chamber, the reaction chamber having a solid support.

12. The apparatus of claim 11, wherein the support comprises a resin.

13. The apparatus of claim 11, wherein the support is a different substrate selected from the group consisting of gold, gold nanoparticles, plasmonic surfaces, and other chip-based sensor technologies that are introduced to various biosensors without the need for separation from the support substrate.

14. The apparatus of claim 1, wherein the apparatus comprises a conduit for transporting the reaction fluid from the reaction chamber to a used reagent collection chamber, and a conduit for transporting the reaction fluid from the used reagent collection chamber to a distillation component, the distillation component having a heating element.

15. The apparatus of claim 14, wherein the heating element heats the distillation component to a specified temperature to separate the reagents in the reaction mixture for future use.

16. The apparatus of claim 15, wherein the distillation separates dimethylformamide (153° C. boiling point), N-methylpiperidine (105° C. boiling point), dichloromethane (39.6° C. boiling point), chloroform (61.2° C. boiling point), acetonitrile (82° C. boiling point), hexafluoro-2-propanol (58.2° C. boiling point), ether (35° C. boiling point), acetone (56° C. boiling point), methanol (65° C. boiling point), tetahydrofuran (66° C. boiling point), hexane (69° C. boiling point), ethyl acetate (77° C. boiling point), N,N-diisopropylethylamine (127° C. boiling point), hydrazine (114° C. boiling point), TFA (72.4° C. boiling point), pyrazole-1-carboxamide (186-188° C. boiling point), or water (100° C. boiling point), toluene (111° C. boiling point), pyridine (115° C. boiling point), acetic acid (118° C. boiling point), dimethylsulfoxide (189° C. boiling point), from one or more reaction fluids in series or parallel for subsequent re-use.

17. The apparatus of claim 1, wherein the apparatus further comprises an in-line purification component.

18. The apparatus of claim 17, wherein the in-line purification component is a high-performance liquid chromatography (HPLC) system.

19. The apparatus of claim 18, wherein the HPLC system includes a plurality of pumps and a plurality of varian switches.

20. The apparatus of claim 1, wherein the apparatus further comprises an in-line lyophilization component, wherein the in-line lyophilization component is used to lyophilize a polymer product at varying stages of synthesis.

21. (canceled)

22. A method of large-scale synthesis of a polymer comprising coupling the polymer to a target substrate using a synthetic staple, wherein the target substrate is a protein, a synthetic product, a nucleic acid, or a biologic product.

23. (canceled)

24. (canceled)
